(19) United States
(12) Patent Application Publication
(10) Pub. No.: US 2020/0020440 A1
(43) Pub. Date: Jan. 16, 2020

(54) COMPUTER-ASSIST METHOD USING DISTRIBUTED LEDGER TECHNOLOGY FOR OPERATING AND MANAGING AN ENTERPRISE

(71) Applicant: Appley Health, Inc., Dade City, FL (US)

(72) Inventor: Gerald W. Beinhauer, Dade City, FL (US)

(21) Appl. No.: 16/510,452

(22) Filed: Jul. 12, 2019

Related U.S. Application Data
(60) Provisional application No. 62/697,368, filed on Jul. 12, 2018.

(51) Int. Cl. G16H 40/20; G06F 16/27; G06Q 50/26; H04L 29/08

(52) U.S. Cl. CPC G16H 40/20; G06F 16/27; H04L 67/141; H04L 67/02; H04L 67/306; G06Q 50/265

(57) ABSTRACT

The present invention generally relates to systems, methods, and non-transitory computer-readable media for processing information to generate deidentified information. In general, de-identified information includes information that does not contain personally identifiable elements that may identify or reasonably be used to identify an individual. The invention utilizes Distributed Ledger Technology to provide a secure mechanism to operating and managing an enterprise.

### CROSS-REFERENCE TO RELATED APPLICATION

This application claims priority from U.S. Provisional Patent Application No. 62/697,368, filed Jul. 12, 2018, titled COMPUTER-ASSIST METHOD USING DISTRIBUTED LEDGER TECHNOLOGY FOR OPERATING AND MANAGING AN ENTERPRISE which is hereby incorporated by reference herein for all purposes.

### BACKGROUND OF THE INVENTION

### 1) Field of the Invention

The invention relates to systems, methods, and non-transitory computer-readable media for processing information to generate de-identified information.

### 2) Description of Related Art

Service providers, retailers, and other businesses collect large amounts of information about their customers and the general public. This information may be used for internal business purposes or aggregated and sold to third parties for marketing and/or analytics activities. Accordingly, customer information has become a commodity. However, the collection of customer information has raised privacy concerns. In response, businesses have pledged to maintain customer anonymity and to protect their information against unauthorized use. In addition, governmental and regulatory bodies have proscribed rules for the collection, management, and sharing of collected information. In the field of healthcare, patient information is regulated according to the federal Health Insurance Portability and Accountability Act of 1996, i.e., HIPAA. Information collected by financial service providers about their customers is regulated according to the Gramm-Leach-Bliley Act, i.e., GLBA or Financial Services Modernization Act of 1999.

Exchanges of patient, provider and payor data using computing systems are often unsecure and prone to undesired alterations. If a particular exchange is compromised, it may be difficult to detect that the specific transaction is compromised. This leads to significant losses in terms of resources, e.g., money, human effort, and time.

In many scenarios, patient, provider and payor data is intercepted or copied by hacking the “secured database”, the source of the hacking can arise from a middle man that handles the exchange. As an example, after booking a hotel room, an individual would need to interact with the desk attendant to pick up the keys to the hotel room. Here, the desk attendant can readily switch the hotel room to a smaller or less desirable hotel room than the one that was booked. Such a switch may not be readily evident to the individual. Thus, there is a need for ensuring safe transactions through the removal of the middle man that handles the exchanges.

The prior art includes U.S. Patent Application Publication No. 2018/0114594 to Bessette et al. which describes a secure methodology to capture and store a patient record so that it is secure, U.S. Patent Application Publication No. 2018/0191714 to Jentzsch et. al. which describes a means of leveraging decentralized blockchain for the storage of information, and U.S. Patent Application Publication No. 2018/0082024 to Cubera.

It should be noted that one focus of data protection efforts is the replacement of the expensive method of utilizing de-identifying information so that it cannot be used to identify an individual, business, or other entity to which the data pertains. In general, current de-identified information does not contain personally identifiable elements that identify or may reasonably be used to identify an entity. Therefore, conventional de-identification methods often allow for de-identified information to be reverted to identifiable information and/or allow for the indirect identification of individuals associated with the de-identified information through minimal effort. Such de-identification methods do not provide adequate protection and, depending on the field of use, may also not comply with applicable laws or regulations. In addition, de-identified data generated through conventional techniques that comply with applicable standards are unsuitable for the robust data aggregation and analytical processing sought by data consumers. For example, certain standards require that de-identified data not include dates or times associated with the information that may be used to identify individuals or entities. As such, data consumers do not have the ability to maintain or access chronological or duration information for such de-identified data. Accordingly, entities that process and/or seek to distribute information collected from customers or the general public would benefit from a system that is secure and capable of storing information such that it sufficiently protects the identity of individuals while allowing for the comprehensive application of data aggregation and analytical processing techniques and the ability to obtain chronology or duration information for the de-identified information. However, such deidentification processes are difficult to operate and manage.

Additionally, a critical relationship is the fundamental relationship in healthcare which is the interaction between two parties: the patient and the physician. Over time, as the healthcare system has expanded, the complexity of processes and events that take place in order to deliver a growing range of products and services to these parties, resulted in an abundance of intermediaries in the market. Typically, the sustainability of intermediaries depends upon their continued ability to deliver significant added value, and often to serve as a facilitator of trust between the two interacting parties. In healthcare; however, most intermediaries have not delivered a level of value to justify their added cost, but they still have managed to position themselves into the equation.

Over the years, because of their strategic positioning, and because there weren't alternative methods to fill the gap that they may fill, these intermediaries have continued to grow and solidify their presence. However, for the first time in this industry, the creation of a new technology called Distributed Ledger Technology (“DLT”) has finally opened the door for disintermediation and is paving the way for slicing significant cost from healthcare.

In its current state, DLT has two primary variants, Public, an open network where anyone can participate without verification or validation, and Private, a closed network where the rules related to participation such as identity verification are determined by the members. Both contain certain key attributes that establish the foundation for its ability to disintermediate third-parties. As its name reveals, DLT involves the establishment of a distributed ledger, or record, that is replicated across a network of parties. No one single party has control over the record, and any attempt to change the record, is immediately identifiable by all parties. This establishes a fully redundant, reliable, immutable instance of the record, with no single point of failure. This enables DLT to serve as a facilitator of trust between transacting parties, because any attempt to change the terms of an agreement would be easily identifiable and trackable across all instances of the ledger, by all participants.

The ability for a record to be stored on the ledger is facilitated by the concept of consensus. There are several types of consensus mechanisms that are employed by different DLT systems. In general, consensus defines the rules and methods by which all participants of the DLT system agree to permit records to be stored on the ledger. Finally, DLT includes high level cryptographic mechanisms that positions it among the most secure solutions to date.

The blockchain/DLT has proven useful for financial transactions and similar types of transactions between various participants. Blockchains are append-only databases of transactions, which are shared and replicated with a consensus algorithm to resolve distributed processing conflicts and prove the validity of the transactions. Each transaction signs and builds on the prior transaction to form an accurate accounting of ownership and value. Each approved transaction becomes part of the chain and is shared among multiple entities, such as enterprise organizations. A ledger model can be quickly adapted for other purposes related to tracking and monitoring user activities, especially, high transaction volume systems. These adaptations may undergo interchange settlement lag, pegged currency of transactions and high-speed negotiations.

Sidechains were developed to address issues with interchange settlement lag, pegged currency of transactions and high-speed negotiation. These secondary chains quickly clear transactions and speed the proof-of-work process. While these secondary chains speed transactions, the overall validation chain could still be further optimized in real-time transactions.

In a self-funded employer model, an insurance carrier (“carrier”) is often positioned in between the provider and the self-funded employer (“Payor”). Although this structure is not desirable in the market today, because there are significant costs added by the carrier, it does help to alleviate the non-payment risk to the provider. Because the provider is contracted with the insurance carrier directly to deliver services, the likelihood that the insurance carrier goes out of business is very small, therefore the non-payment risk if small. When the provider delivers a service and submits his/her medical billing claim to the carrier, the carrier pays the provider and then the carrier charges the employer. The carrier does take on some of this risk. Carriers typically require the employer to keep at least ninety days of estimated claims payments in their trust account to reduce some of this risk from the carrier.

In disintermediation and direct peer-to-peer relationships often levering DLT, the non-payment risk between the parties can become a factor. Instead of a carrier, which are usually relatively larger than the self-funded employer for whom they service, being in the middle, the self-funded employer to provider relationship is direct. In other words, if the provider delivers a service to one of the employer's plan members, i.e., employees and their families, then the likelihood that the employer can't or doesn't pay is much greater to the provider.

Ubiquitous access to healthcare data has always been talked and dreamt about, but it has never come to fruition. It is well known that the ability to achieve a single aggregated view of a patient's longitudinal record, i.e., a medical record that encompasses all places of care that spans the lifetime, could open up a new world of actionable insights to improve health, wellness, and lifespan. Unfortunately to this day, most healthcare data exists in distinct data silos, with little to no interconnectivity between them to establish an aggregated longitudinal record. Some of this problem has been driven by technical limitations, but most has been related to business tactics that drive the concept of “data blocking.” There has been a tremendous push, both politically and from an industry technical perspective to push toward achieving data interoperability through API connectivity.

In addition, there is no standardized patient record number system. Each electronic or paper-based system of a provider typically employs a unique patient medical record numbers. Without a standardized, national, or global, patient record numbering system, patient record lookup, matching, and merging takes place based on other patient identifiable queries, e.g., name, dob, zip, and address. Typically, the manual data entry at each location by staff introduces variations in the patient identification making record matching and merging very difficult to impossible.

There is a significant shift in the industry today toward direct contracting for healthcare services between self-funded employer health plans and providers, e.g., doctors, doctor groups, hospitals, health systems, labs, and imaging. The reason for this is it reduces costs and enables control for the employer and increases reimbursements and speed to payment for the provider. However, the manner in which this takes place today is through traditional one-to-one, paper contracts between providers and employers. Each employer enters into a contract with each provider, or vice versa. This is not scalable and does not introduce market dynamics such as pricing stabilization and standardization.

In today's healthcare market, contracting, network participation, and “in-network” vs. “out-of-network” status is formed on a one-to-one basis. Therefore, the typical provider directory that lists the providers who are participating under a contract or payer's plan, are not very dynamic. Furthermore, today's antiquated contracting approach is not very granular. Typically, the provider is either in-network or out-of-network. There is limited ability to drill down to a much more granular level such as excluding certain locations, specific doctors, and specific procedures.

Historically for those entities who wish to build a network of healthcare providers, this would require different forms of payers, this was a very time intense, manual and tedious process. It typically involved “feet on the street” repetitive phone calls, ongoing tenacious sales, and significant experience and manpower. The approach is typically done on a market by market basis and takes months to years to accomplish. There are also no economies of scale afforded to the current approach and no dynamic or market effect employed, e.g., if one entity builds a network in a particular area, another entity would do the same.

When healthcare providers deliver services, some level of verification of their medical credentials, e.g., state medical license, board certification, malpractice insurance, and malpractice claim history, is typically required from payers, hospitals, the medical groups that employ them, and even the self-funded plans that contract with them. Today, this process usually involves a third-party service who performs this task for a fee and serves as the intermediary to facilitate trust between the parties. This approach requires that the provider input, enter, and/or upload their sensitive information to this third-party, who then stores it, assesses it, and determines if the provider has adequate information to be considered “credentialed.” This third-party intermediary creates an unnecessary added level of cost, complexity, and security risk, that does not need to exist through the use of DLT and smart contract technology.

Therefore, what is needed in the art is a way to improve the security and enhance the interaction between parties and reduce the associated costs.

What is also needed in the art is a secure method to store and transfer health record data.

What is also needed in the art is a method to support relationships between multiple parties.

What is also needed in the art is a dynamic, scalable and customizable method to support contracting with providers and creating a network to access these providers.

### BRIEF SUMMARY OF THE INVENTION

The invention in one form is directed to a method of building a network of users. The method includes providing a platform, inputting information about a user, saving the info to the platform, allowing for querying the platform for other users, allowing a user to invite other users to join their network, generating one or more communications on the platform that are used to facilitate the invitation, requesting an acceptance of the invitation from the other user, and sending one or more notifications to the user.

The platform includes a distributed ledger, a web-based portal that is configured for one or more connections, and a computer system in connection with the web-based portal. The platform further includes another computer system that has an application programming interface and may be in connection with the web-based portal. The platform may also include a session controller in connection with the web-based portal, one or more web-based engines in connection with the web-based portal, and one or more web-based databases in connection with the web-based portal.

The invention in another form is directed to a method of facilitating direct contracting. The method includes providing a platform, establishing a relationship between users of the platform, selecting a user profile from a list on the platform, reviewing one or more contracts associated with the user profile, and selecting one or more contracts.

The platform includes a distributed ledger, a web-based portal that is configured for one or more connections, and a computer system in connection with the web-based portal. The platform further includes another computer system that has an application programming interface and may be in connection with the web-based portal. The platform may also include a session controller in connection with the web-based portal, one or more web-based engines in connection with the web-based portal, and one or more web-based databases in connection with the web-based portal.

In yet another form, the invention is a system for managing information. The system includes a distributed ledger, a web-based portal that is configured for one or more connections, and a computer system that may be in connection with the web-based portal. The system may also include another computer system that has an application programming interface. The other computer system may be in connection with the web-based portal. There is also provided a session controller that may be in connection with the web-based portal, one or more web-based engines that may be in connection with the web-based portal, and one or more web-based databases that may be in connection with the web-based portal.

An advantage of the present invention enables users to build and operate their own network of healthcare providers through a computer-assisted distributed ledger-based platform (“Platform”).

Another advantage of the present invention enables providers to directly serve non-conventional payors through the Platform.

Another advantage of the present invention facilitates automated provider credentialing through the Platform.

Another advantage of the present invention enables users to contract directly through the Platform.

Another advantage of the present invention facilitates an automated electronic medical billing claims processing and digital payment through the Platform.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 describes an information and data flow for an embodiment of a Method and Process to Build a Network of Providers;

FIG. 2 describes the continuation of the information and data flow for an embodiment of a Method and Process to Build a Network of Providers;

FIG. 3 depicts a flow diagram for an embodiment of a Method and Process to Build a Network of Providers to determine if the payor is a legal entity or an individual;

FIG. 4 depicts a flow diagram for an embodiment of a Method and Process to Build a Network of Providers;

FIG. 5 is a continuation of the embodiment shown in FIG. 4;

FIG. 6 depicts a flow diagram for an embodiment of a Method and Process to Build a Network of Providers;

FIG. 7 is a continuation of the embodiment shown in FIG. 6;

FIG. 8 is a continuation of the embodiment shown in FIG. 7;

FIG. 9 describes an embodiment of a Method and Process to Build a Network of Payors;

FIG. 10 describes an embodiment of a Method and Process to Facilitate Automated Provider Credentialing;

FIG. 11 describes an embodiment of a Method and Process to Facilitate Direct Contracting with Automated Oversight; and

FIG. 12 describes an embodiment of a Method and Process to Facilitate Automated Electronic Claims Processing and Payment.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

In this application the use of the Blockchain, Distributed Ledger Technology or DLT are used interchangeably.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

The following applications, U.S. Patent Application Publication No. 2018/0114594 Patient-Centric Health Record System and related methods, published Apr. 26, 2018, which describes a secure methodology to capture and store a patient record so that it is secure, U.S. Patent Application Publication No. 2018/0191714, Block Chain Enabled Service Provider System, published Jul. 5,2018, which describes leveraging decentralized blockchain for the storage of information, U.S. Patent Application Publication No. 2016/0306999, System, Methods, and computer-readable media for de-identifying information, published Oct. 20, 2016, U.S. Patent Application Publication No. 2018/0082024, Secure Distributed Patient Consent and Information Management, published Mar. 22, 2018. All publications, including but not limited to patient, provider and payor applications, cited in this specification are herein incorporated by reference as if each individual publication were specifically and individually indicated to be incorporated by reference herein as though fully set forth.

The illustrative embodiments of the present invention provide mechanisms that implement secure distributed patient, provider and payor information management which address the competing needs in the health care industry for efficient access to electronic records by authorized parties while maintaining protection of privacy of related information and complying with governing regulations. The terms patient, provider and payor can be used interchangeably throughout the application.

The mechanisms of the illustrative embodiments implement DLT/blockchain technology to manage a user's information, e.g., patient, provider and payor and distribution of the user's information, e.g., patient, provider and payor, based on the user's consent. The user's data, e.g., patient, provider and payor, always follows the user' information and records of transactions and their associated linkages and is maintained in a tamper-proof and immutable blockchain/DLT data structure.

In general, de-identified information includes information that does not contain personally identifiable elements that may identify or reasonably be used to identify an individual. This improved blockchain/DLT mechanism operates in conjunction with a master record (“MR”). The MR may be any type of record including a Master Patient Index. The MR which when viewed with respect to the instant invention is a record that requires a secure storage and distribution methodology. The MR component must enable the exchange of user's data, e.g., patient, provider and payor information, among health providers, once the data has been loaded, verified and access has been granted by the user, e.g., patient, provider and payor as applicable. Only those entities for which a user's authorization, e.g., patient, provider and payor and the electronic document or data structure that indicates authorization has been granted will be given a record locator that identifies the location of a particular portion of information. Once provided with a record locator from the MR, the entity that is granted access can directly request information from another entity, where the information resides as indicated by the record locator, or the mechanisms of the illustrative embodiments may operate as an intermediary data hub with regard to secure information transfer, depending on the desired implementation.

Although DLT possesses the technical characteristics to serve as a foundational for disintermediation in the healthcare market, the methods and techniques related its use in administering, operating, and managing an enterprise have yet to be fully implemented. The benefits for the use of DLT in healthcare can be appreciated in several key areas related to direct contracting, network building, non-conventional payor-provider operations, and process automation.

With the mechanisms of the illustrative embodiments, many benefits and use cases are enabled that provide significant advantages over existing mechanisms. For example, using the improved blockchain/DLT and index mechanisms of the illustrative embodiments, users, e.g., patient, provider and payor, are always able to determine where their information resides and to whom the entity who has been granted access, and to what portions of the information access was granted. This further permits distributed storage of the information rather than having to have a centralized repository.

Secure distributed ledger technologies such as a blockchain/DLT, where identical and tamper-proof copies of the ledger are maintained, this structure provides a higher level of trust than single ‘trusted third party’ solutions, and such structure assures only authorized parties will receive and can act on valid, secure, fully trusted information. Furthermore, through the blockchain/DLT or other ledger-based mechanisms of the illustrative embodiments, an immutable store of transactions of information, linking information being transacted and the entities involved in the transaction is provided.

It should be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general-purpose hardware, a procedure or method for executing the functions, or a combination of any of the above.

The current invention describes several novel computer-assisted methods and techniques that utilize Blockchain/Digital Ledger Technology (“DLT”) in a practical and novel way to achieve a competitive advantage in the marketplace and deliver tangible value.

Specifically, the invention describes a computer-assisted method related to operating and managing an enterprise, enabling: 1) payors of healthcare to build a network of healthcare providers, 2) healthcare providers to build a network of payors, 3) multiple parties to engage under direct contractual relationships with automated oversight, 4) electronic medical billing claims processing and digital payment between parties, and 5) all participating payors and providers to benefit from the ecosystem that is created through the efforts of each individual party.

For clarity in the current invention, the term payor may include, but is not limited to, any individual person or legal entity, and the term Provider or Healthcare Provider may include, but is not limited to, any individual person or legal entity.

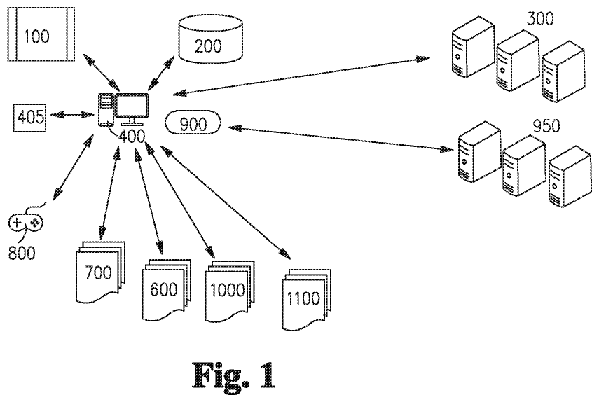

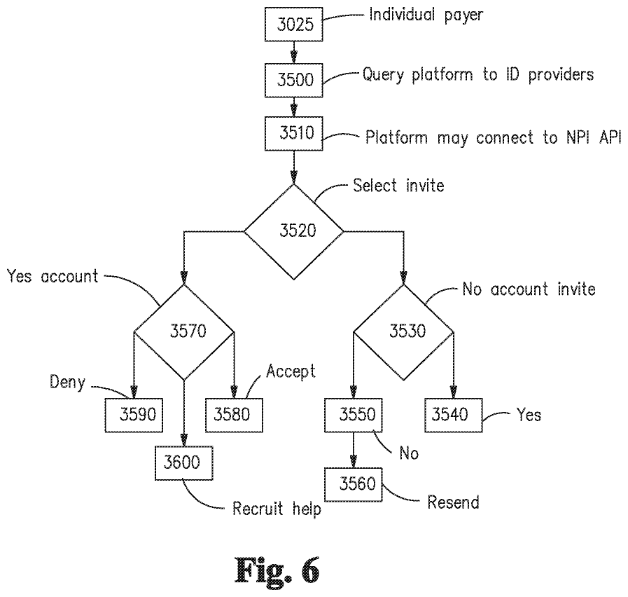

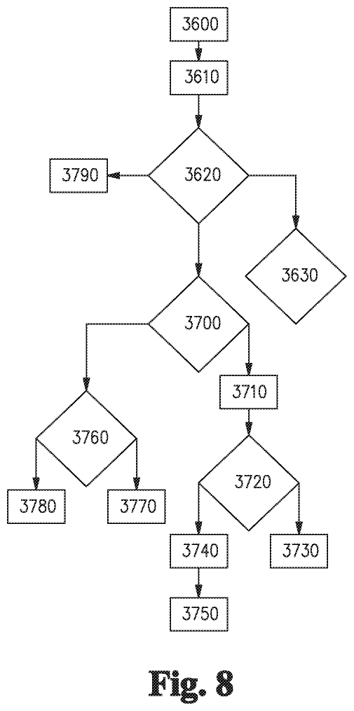

With respect to FIGS. 1-12, which are described in this application, such figures and accompanying specification are to be interpreted with respect to utilization of distributed ledger technology/blockchain technology.

Referring now to the drawings, and more particularly to FIG. 1, there is shown an integrated platform (“Platform”) that incorporates several components and information sources: distributed ledger technology software 100, third-party cloud-based distributed computer system 200, access to a global network of interconnected computers 300, a web-based portal 400, a business process and rules engine 405, an integration engine 500, an interface engine, a clinical data repository 600, a medical record and claims processing engine 700, a session controller 800, API 900 connectivity to the National Provider Identifier system 950, healthcare services pricing data from the Centers for Medicare and Medicaid Services (“CMS”) 1000 and other sources, payor and provider contact data from third-party sources.

The present invention employs a novel method and technique for conducting business that delivers tangible value in the form or cost savings to payors, by enabling them to build and operate their own network of healthcare providers through a computer-assisted distributed ledger-based platform.

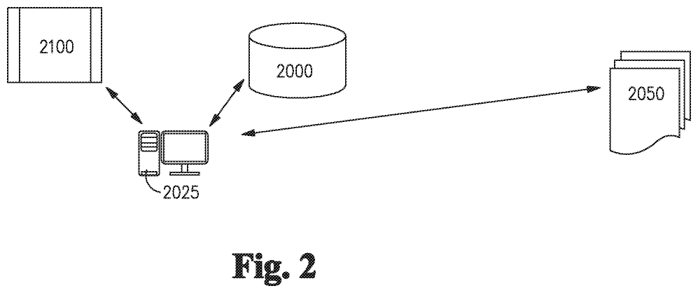

Referring now to FIGS. 2-8 there is shown an embodiment of a Method and Process to Build a Network of Providers.

Shown in FIG. 2, a payor establishes an account 2100 on the Platform 2000 using computer 2025. The payor may also have an established account on the Platform and wants to build or expand their provider network 2050.

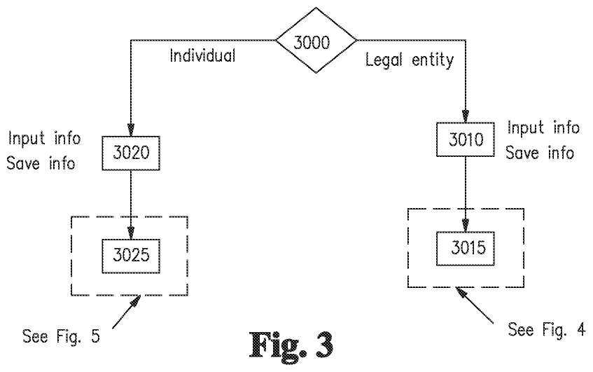

FIG. 3 continues the process if the payor is a legal entity that facilitates healthcare for other legal entities or individuals. Step 3010, the payor inputs and saves the information of those legal entities and/or individuals and dependents for whom it facilitates healthcare and stores it on the Platform. Step 3020, the payor inputs and saves the information for whom the payor is responsible for facilitating healthcare and stores it on the Platform.

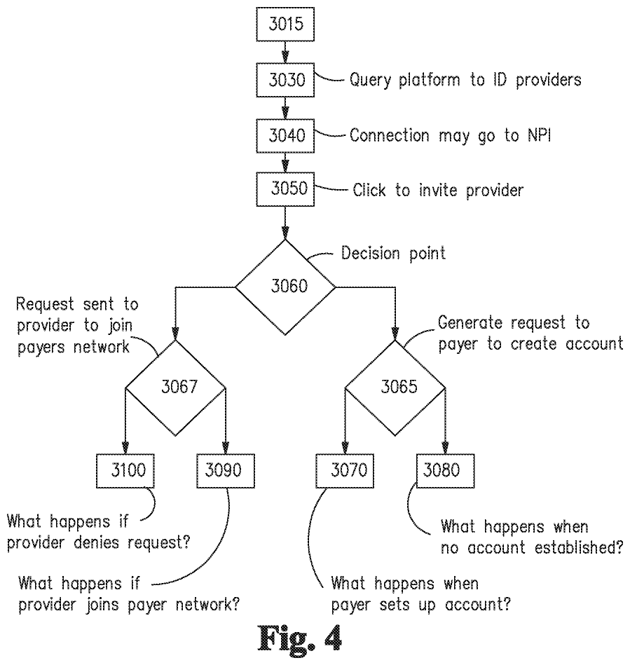

FIG. 4 continues the process if the payor is a legal entity that facilitates healthcare for other legal entities or individuals. Step 3030, the payor directly queries the Platform to identify those providers that the payor would like to invite to join the payor's network and clicks to invite those providers to join the payor's network and contract with the payor. Step 3040, in some embodiments, when querying the Platform for providers, the Platform connects to the National Provider Identifier API to facilitate this lookup and result. In other embodiments, the method of querying and identifying those providers with whom the payor would like to invite employs a marketplace environment with certain filters, search criteria, and reviews that the payor can utilize to make a determination. Step 3050, when clicking to invite providers to join the payor's network and contract with the payor, Step 3060 the system may determine if the provider does not yet have an account established on the Platform. Step 3065, the Platform may generate one or more communications, including an email to the provider, a notification to the Platform administrator, a fax to the provider, and other automated notification methods, to facilitate the invitation. Step 3070, if the provider responds to the invitation by creating an account on the Platform and accepting the payor's invitation within seventy-two hours, the payor may be notified of the provider's acceptance. Step 3080, if the provider does not create an account on the Platform within seventy-two hours, the Platform administrator may be notified again by the Platform to reach out to the provider. The provider's decision of acceptance or denial of the payor's request may be sent as a notification to the payor.

Returning to Step 3060, the system may determine that the provider does have an established account on the Platform. Step 3067, the Platform may send a notification to the provider's account asking if they accept the payor's invitation to join the payor's provider network. Step 3090, if the provider accepts the invitation, the Platform may send a notification to the payor's account, and the relationship is established. Step 3100, if the provider denies the invitation, the Platform may send a notification to the payor's account, advising of the denial.

Step 3110, the payor may select to Recruit help from the legal entities and/or individuals for whom the payor facilitates healthcare, whose information was previously inputted and saved in Step 3000.

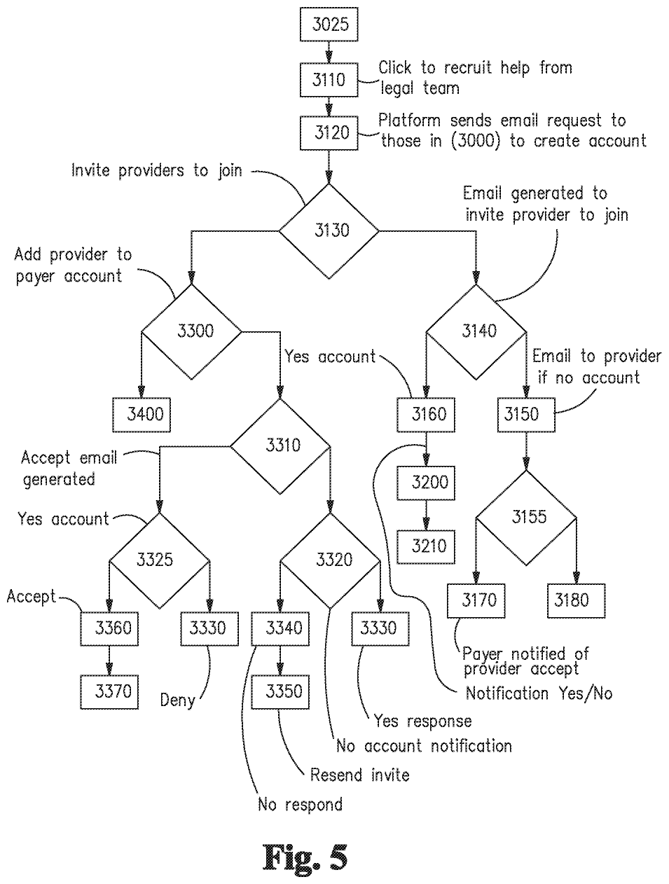

Shown now in FIG. 5, Step 3120, when clicking to recruit help, the Platform may send an email invitation to those individuals whose information was previously inputted and saved. The Platform may send an email invitation to their email addresses that were stored, asking them to create an account on the Platform, and inputs those providers that they wish to invite to join their provider network. Step 3130, when clicking to invite providers to join the Platform and contract with the payor based on the Platform settings, as determined by the payor, the Platform may either go to Step 3140, generating one or more communications, including an email to the provider, a notification to the Platform administrator, a fax to the provider, and other automated notification methods, to facilitate the invitation. The Platform may also go to Step 3150, if the provider does not yet have an account established on the Platform, the Platform may generate one or more communications, including an email to the provider, a notification to the Platform administrator, a fax to the provider, and other automated notification methods, to facilitate the invitation. Step 3170, if the provider responds to the invitation by creating an account on the Platform and accepting the payor's invitation within seventy-two hours, the payor may be notified of the provider's acceptance. Step 3180, if the provider does not create an account on the Platform within seventy-two hours, the Platform administrator may be notified again by the Platform to reach out to the provider. The provider's decision of acceptance or denial of the payor's request may be sent as a notification to the payor.

Referring now to Step 3160, if the provider does have an established account on the Platform, the Platform may send a notification to the provider's account asking if they accept the payor's invitation to join the payor's provider network. Step 3200, if the provider accepts the invitation, the Platform may send a notification to the payor's account, and the relationship is established. Step 3210, if the provider denies the invitation, the Platform may send a notification to the payor's account, advising of the denial.

Step 3300, allows the addition of the provider to the payors account, pending the payor's acceptance or denial to invite the provider to join the network. Step 3310, if the payor accepts the request, the Platform may generate one or more communications, including an email to the provider, a notification to the Platform administrator, a fax to the provider and other automated notification methods to facilitate the invitation. Step 3320, if the provider does not yet have an account established on the Platform, the Platform may generate one or more communications, including an email to the provider, a notification to the Platform administrator, a fax to the provider, and other automated notification methods, to facilitate the invitation. Step 3330, if the provider responds to the invitation by creating an account on the Platform and accepting the payor's invitation within seventy-two hours, the payor may be notified of the provider's acceptance. Step 3340, if the provider does not create an account on the Platform within seventy-two hours, the Platform administrator may be notified again by the Platform to reach out to the provider. Step 3350, the provider's decision of acceptance or denial of the payor's request may be sent as a notification to the payor. Step 3325, if the provider does have an established account on the Platform, the Platform may send a notification to the provider's account asking if they accept the payor's invitation to join the payor's provider network. Step 3360, if the provider accepts the invitation, the Platform may send a notification to the payor's account, and the relationship is established. Step 3370, if the provider denies the invitation, the Platform may send a notification to the payor's account, advising of the denial. Step 3400, if the payor denies the request, the Platform may generate one or more communications, including a message back to notify the legal entity or individual who submitted the provider request of the payor's denial of their request.

If the payor is an individual, the payor in Step 3500, may query the Platform to identify the providers that the individual would like to invite to join the individual's provider network and click to invite those providers to join the Platform and contract with the payor. Step 3510, in some embodiments, when querying the Platform for providers, the Platform is connecting to the National Provider Identifier API to facilitate this lookup and result. In other embodiments, the method of querying and identifying those payors with whom the payor would like to invite includes a marketplace environment with certain filters, search criteria, and reviews that the individual can utilize to make a determination. Step 3520, when clicking to invite providers to join the Platform and contract with the payor, the Platform determines if the provider has an account.

Step 3530, if the provider does not yet have an account established on the Platform, the Platform may generate one or more communications, including an email to the provider, a notification to the Platform administrator, a fax to the provider, and other automated notification methods, to facilitate the invitation. Step 3540, if the provider responds to the invitation by creating an account on the Platform and accepting the payor's invitation within seventy-two hours, the payor may be notified of the provider's acceptance. Step 3550, if the provider does not create an account on the Platform within seventy-two hours, the Platform administrator may be notified again by the Platform to reach out to the provider. Step 3560, the provider's decision of acceptance or denial of the payor's request may be sent as a notification to the payor.

Step 3570, if the provider does have an established account on the Platform, the Platform may send a notification to the provider's account asking if they accept the payor's invitation to join the payor's provider network. Step 3580, if the provider accepts the invitation, the Platform may send a notification to the payor's account, and the relationship is established. Step 3590, if the provider denies the invitation, the Platform may send a notification to the payor's account, advising of the denial.

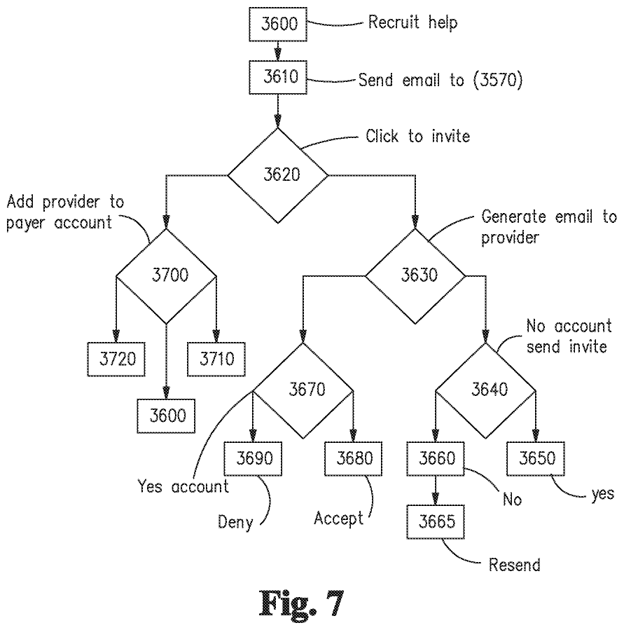

As shown in FIG. 7, the payor may also request help. Step 3600, the payor clicks to recruit help from those for whom the payor facilitates healthcare, whose information the payor may have previously inputted and saved. Step 3610, when clicking to recruit help, the Platform may send an email invitation to those individuals whose information that was inputted and saved. The Platform may send an email invitation to their email addresses that were stored and ask them to also create an account on the Platform and input those providers that they wish to invite to join their provider network. Step 3620, when clicking to invite providers to join the Platform and contract with the payor, based on the Platform settings, as determined by the payor, the Platform determines if the provider has an account. Step 3630, the Platform may then generate one or more communications, including an email to the provider, a notification to the Platform administrator, a fax to the provider, and other automated notification methods, to facilitate the invitation.

Step 3640, if the provider does not yet have an account established on the Platform, the Platform may generate one or more communications, including an email to the provider, a notification to the Platform administrator, a fax to the provider, and other automated notification methods, to further facilitate the invitation. Step 3650, if the provider responds to the invitation by creating an account on the Platform and accepting the payor's invitation within seventy-two hours, the payor may be notified of the provider's acceptance. Step 3660, if the provider does not create an account on the Platform within seventy-two hours, the Platform administrator may be notified again by the Platform to reach out to the provider. Step 3665, the provider's decision of acceptance or denial of the payor's request may be sent as a notification to the payor.

Step 3670, if the provider does have an established account on the Platform, the Platform may send a notification to the provider's account asking if they accept the payor's invitation to join the payor's provider network. Step 3680, if the provider accepts the invitation, the Platform may send a notification to the payor's account, and the relationship is established. Step 3690, if the provider denies the invitation, the Platform may send a notification to the payor's account, advising of the denial. Step 3700, the Platform adds the provider to the payors account pending the payor's acceptance or denial to invite the provider to join the network. Step 3710, if the payor accepts the request, the Platform may generate one or more communications, including an email to the provider, a notification to the Platform administrator, a fax to the provider and other automated notification methods to facilitate the invitation. Step 3720, if the provider does not yet have an account established on the Platform, the Platform may generate one or more communications, including an email to the provider, a notification to the Platform administrator, a fax to the provider, and other automated notification methods, to facilitate the invitation. Step 3730, if the provider responds to the invitation by creating an account on the Platform and accepting the payor's invitation within seventy-two hours, the payor may be notified of the provider's acceptance. Step 3740, if the provider does not create an account on the Platform within seventy-two hours, the Platform administrator may be notified again by the Platform to reach out to the provider. Step 3750, the provider's decision of acceptance or denial of the payor's request may be sent as a notification to the payor. Step 3760, if the provider does have an established account on the Platform, the Platform may send a notification to the provider's account asking if they accept the payor's invitation to join the payor's provider network. Step 3770, if the provider accepts the invitation, the Platform may send a notification to the payor's account, and the relationship is established. Step 3780, if the provider denies the invitation, the Platform may send a notification to the payor's account, advising of the denial. Step 3790, if the payor denies the request, the Platform may generate one or more communications, including a message back to notify the legal entity or individual who submitted the provider request of the payor's denial of their request.

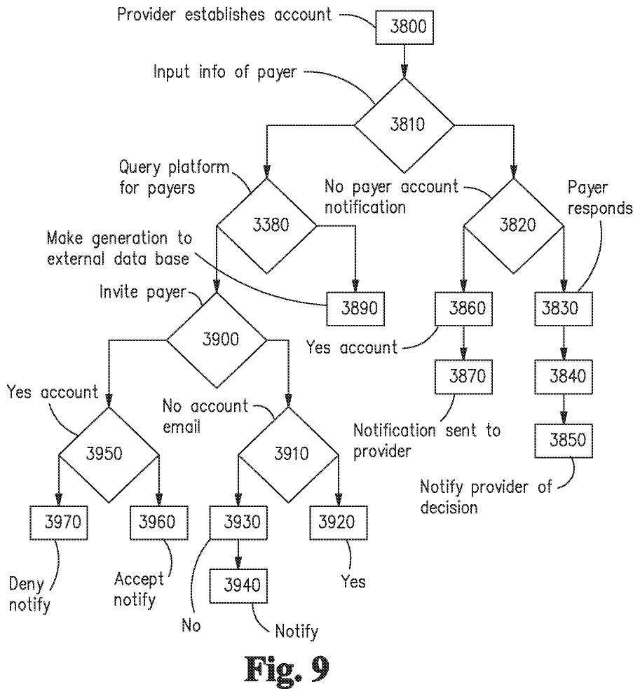

In another embodiment of the present invention, there is a method and process for providers to establish their own network of payors using the Platform. Now referring to FIG. 9, there is shown an embodiment of the present invention that employs a novel method and technique for conducting business that delivers tangible value to providers in the form of increased revenue, service expansion, ability to expand market penetration, and competitive advantage, by enabling providers to directly serve non-conventional payors through the Platform. For clarity, non-conventional payors may include, but are not limited to, self-funded entities, i.e., employers, municipalities, schools and colleges, individuals, and other legal entities.

Step 3800, a provider may establish an account on the Platform or may already have an established account on the Platform where the provider wants to build or expand their network of payors. Step 3810, the provider inputs and saves the information of the payor with whom the provider wishes to invite to its network onto the Platform.

Step 3820, if the payor does not yet have an account established on the Platform, the Platform may generate one or more communications, including an email to the payor, a notification to the Platform administrator, a fax to the payor, and other automated notification methods, to facilitate the invitation. Step 3830, if the payor responds to the invitation by creating an account on the Platform and accepting the provider's invitation within seventy-two hours, the provider may be notified of the payor's acceptance. Step 3840, if the payor does not create an account on the Platform within seventy-two hours, the Platform administrator may be notified again by the Platform to reach out to the payor. Step 3850, the payor's decision of acceptance or denial of the provider's request may be sent as a notification to the provider.

Step 3860, if the payor does have an established account on the Platform, the Platform may send a notification to the payor's account asking if they accept the provider's invitation to join the provider's network. Step 3870, if the payor accepts the invitation, the Platform may send a notification to the provider's account, and the relationship is established.

The provider may also run a query on the Platform to identify payors. Step 3880, the provider queries the Platform to identify those payors that the provider would like to invite to join the provider's network and clicks to invite those payors to join the provider's network and contract with the provider. Step 3890, in some embodiments, when querying the Platform for payors, the Platform connects to data sources that includes payor information. In other embodiments, the method of querying and identifying those payors with whom the provider would like to invite may employ a marketplace environment with certain filters, search criteria, and reviews that the provider can utilize to make a determination. Step 3900, when clicking to invite payors to join the provider's network and contract with the provider, the Platform may determine if the payor has an account.

Step 3910, if the payor does not yet have an account established on the Platform, the Platform may generate one or more communications, including an email to the payor, a notification to the Platform administrator, a fax to the payor, and other automated notification methods, to facilitate the invitation. Step 3920, if the payor responds to the invitation by creating an account on the Platform and accepting the provider's invitation within seventy-two hours, the provider may be notified of the payor's acceptance. Step 3930, if the payor does not create an account on the Platform within seventy-two hours, the Platform administrator may be notified again by the Platform to reach out to the payor. Step 3940, the payor's decision of acceptance or denial of the provider's request may be sent as a notification to the provider. Step 3950, if the payor does have an established account on the Platform, the Platform may send a notification to the payor's account asking if they accept the provider's invitation to join the provider's network. Step 3960, if the payor accepts the invitation, the Platform may send a notification to the provider's account, and the relationship is established. Step 3970, if the payor denies the invitation, the Platform may send a notification to the provider's account, advising of the denial.

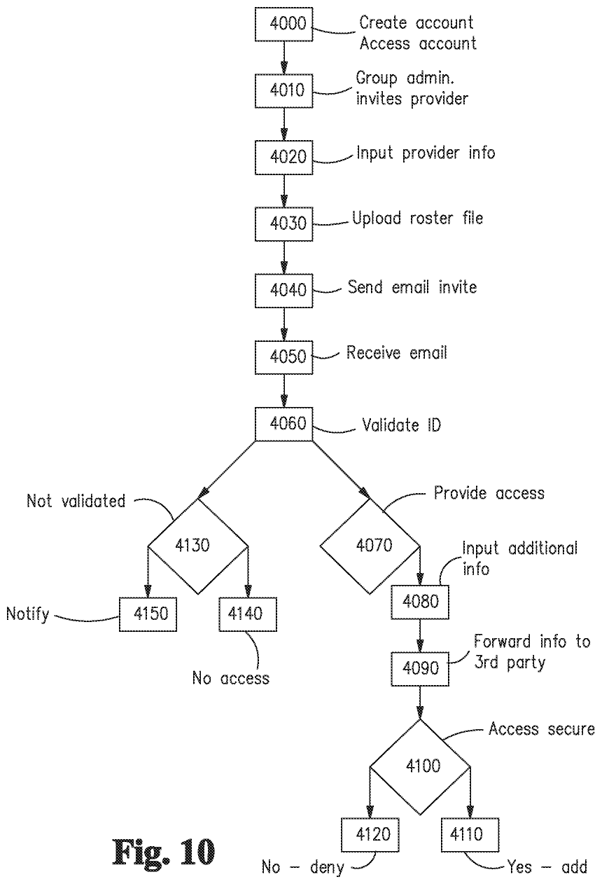

Referring now to FIG. 10, there is shown an embodiment of a Method and Process to Facilitate Automated Provider Credentialing. The invention employs a novel method and technique for conducting business that delivers tangible value in the form of cost and time savings to payors and increased revenue and timeliness of payment to providers, by facilitating an automated provider credentials verification process through the Platform.

Step 4000, a provider creates or has an established account on the Platform and wishes to invite and credential individual providers under their provider group. Step 4010, provider group administrator (“Admin”) invites individual providers to join the provider group account. Step 4020, Admin may input and save to the Platform each provider's national provider identifier number and active/valid email address. Step 4030, the Admin may also upload a file that contains a roster of this information. Step 4040, the Platform may then send one or more communications, including an email invitation to the email addresses that were entered into the Platform, requesting that the provider create an account on the provider group account. Step 4050, the individual provider receives the communication, e.g., email, and clicks on the link therein. Step 4060, the individual provider may be required to validate his/her identity a verification process that may reside on the Platform or provided through a third-party identity verification services that is accessed by the Platform with an API. Step 4070, if the individual provider validates his/her identity, then he/she is granted access to the provider group account on the Platform. Step 4080, the provider is asked to input some of his/her information that is needed for verifying his/her credentialing status. Step 4090, once the information is entered into the Platform, the Platform may forward this information to a third-party credentialing service via an API or other methods. The third-party credentialing service may respond by transmitting a credentialing score back to the Platform, with a review of any critical, high, or low issues. Step 4100, the Platform determines using rules set by the Platform administrator, whether or not the score is acceptable to accept the individual onto the Platform. Step 4110, if the score is acceptable, then the individual provider is added to the provider group account, the Admin may be notified, and is eligible to perform functions on the Platform. Step 4120, if the score is unacceptable, the individual provider, the Admin, and the Platform Admin may be notified of the denial and the reasons for this.

Step 4130, if the individual provider is not able to validate his/her identity, then the Platform may perform additional steps. Step 4140, the Platform may not grant access to the provider group account on the platform. Step 4150, the individual provider, the provider group Admin, and the Platform Admin may be notified of the access denial and the reasons for this, and the parties can determine the reason for this denial.

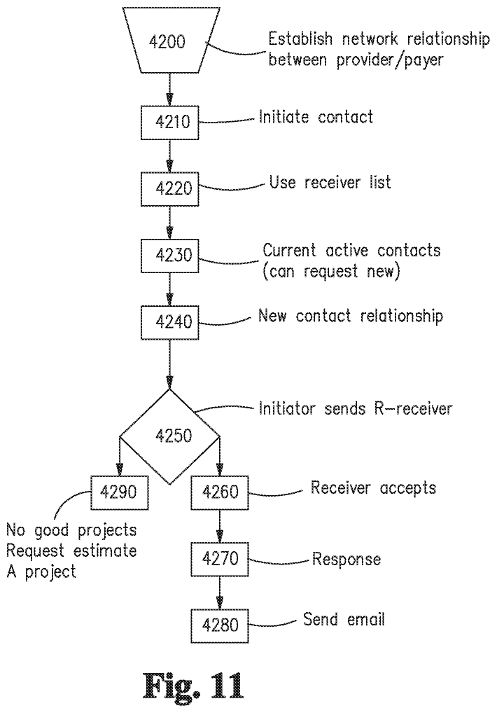

Referring now to FIG. 11, there is shown an embodiment of a Method and Process to Facilitate Direct Contracting with Automated Oversight. The invention employs a novel method and technique for conducting business that delivers tangible value in the form of cost savings to payors and increased revenue and timeliness of payment to providers, by enabling them to contract directly through the Platform.

Step 4200, a payor and a provider have created their respective accounts on the Platform and have established a network relationship between them, e.g., either through a payor to provider approach previously described, or a provider to payor approach also previously described. Step 4210, one of the parties (“Initiator”) desires to enter into a direct contract with the other party (“Receiver”), either as a first contract, or an additional/secondary one. Step 4220, the Initiator clicks on the desired Receiver listed within the respective Platform portal. Step 4230, under the contracts area of the Receiver's profile, the Initiator is presented with a list of any current active contracts that may already exist between the Initiator and the Receiver, and a button to request a new contractual relationship. Step 4240, when the Initiator clicks on the button to request a new contractual relationship, the Initiator is presented with a list of contracts that are currently available on the Platform, an indicator showing if this particular Receiver is already engaged under any of these contracts with other providers, and the ability to request the Platform Admin to initiate a project to develop a new contract to be added to the Platform. Step 4250, when the Initiator chooses to enter into a new contractual relationship with the Receive, and chooses one of the contracts that is listed, the Platform may ask for confirmation of this selection from the Initiator, and when confirmed, may send a notification to the Receiver with a link to the desired contract for review. Step 4260, if the Receiver accepts the request within seventy-two hours, the contract may be immutably stored on the Platform, and the Initiator may be notified. Step 4270, if the Receiver has not responded to the request within seventy-two hours, then the Platform Admin may be notified by the Platform to reach out to the Receiver to determine their decision. Step 4280, if the Receiver denies the request, the Initiator may be notified of the decision. Step 4290, when the initiator does not identify any contracts that are listed on the Platform as being desirable, the Initiator can then submit a request to the Platform Admin to establish a project to create a contract that meets the Initiators desired structure.

One attribute of DLT is the ability to implement “smart contracting.” Smart contracts are programmatic code that auto-runs on a distributed network, based on terms and parameters that two parties agree upon at the onset of the smart contract's initial acceptance and execution between them. Smart contracts often utilize a concept called an “oracle”, i.e., a truth teller, as a third-party to determine certain aspects of a smart contract. Oracles may be used to determine the amount of payment that is owed by one party if the payment amount is variable, for example the London Inter-bank Offered Rate (“LIBOR”) average.

DLT with smart contract functionality, and a few other functions can enable direct relationships between payers and provider with the elimination of the non-payment risk previously discussed.

In one embodiment of a method to determine eligibility, prior authorization, escrow, and claims repricing there is a distributed computing network, with a distributed operating system, running a distributed ledger application with smart contracting functionality (collectively, “Platform”). Defined in the smart contract on this Platform are the requirements of each party in both a pre-service, e.g., before services are delivered by a party, and post-service state, e.g., after services are delivered by a party.

The method may have more than one party, e.g., first party and a second party, such as a self-funded employer and a provider of healthcare services.

The second party may already be engaged under the smart contract on the Platform and is already available to deliver services to any other party on the Platform who also participates in the smart contract. Prior to engaging under the smart contract, a first party is required to deposit a predetermined amount of funds or purchase a predetermined number of digital tokens, and maintain that amount in their account, e.g., digital wallet, on the Platform. The value of predetermined amount or number can be determined by many different factors defined in the smart contract, including but not limited to, the number of members that the first party is covering under its self-funded health plan, or even a claims analytics engine, e.g., a rules based engine, that can forecast future care based on historical claim details.

In a Pre-Service State, after the first party has met the above requirement and is engaged under the smart contract, one of the first party's plan members contacts the second party and requests to be seen by a provider, e.g., a doctor.

The second party may search for and select a first party member on the Platform. The second party may then submit a request for authorization to deliver certain services to a first party member. In doing so, the second party inputs the proposed services to be delivered to a first party member. This may be completed by the second party using their existing practice management and/or billing system having connectivity to the Platform. The Platform may compile this information, and using the previously agreed upon oracle, e.g., Medicare pricing database, determine the amount of payment for the service being requested and other smart contract rules. The Platform may then determine if a first party member is eligible for the service and verify that the amount of payment is available in the first party account, e.g., digital wallet. If approved, the Platform may place the amount of payment for the service in an account, e.g., escrow account, on the Platform. The Platform may then notify the second party of the approval, notify the first party of the service request and approval, and notify a first party member of the approval. Other rules, e.g., time limit for fulfilling the service and location restrictions associated with the service request, can also be defined in the smart contract that can accompany the approval.

If denied, the Platform may notify the first party of the service request and reason for denial, notify the second party of the denial and reason, and notify a first party member of the denial and reason. It is to be understood that the above process typically defines eligibility and request for prior-authorization but may be parsed to only encompass eligibility without a request for authorization.

If the second party delivers a service to the first party, i.e., the plan member of the first party sees the doctor, the second party may submit their medical billing claim (“claim”) to the Platform for the services that were delivered. Included in this claim may be the ID number related to the pre-service state prior-auth. The Platform receives this claim and may then verify that the services that were claimed to be delivered, coincide with the services requested in the pre-service state. The Platform may then re-price the claim based on the contract rate, and then either process the claim or pass the claim to another third party for processing. The Platform may then send the first party member a notification requesting feedback related to their visit with the provider. The Platform may then use the feedback from the first party member as confirmation that the first party member did receive the service from the second party. If the first party member did not confirm the services via participating in the survey above, the Platform may specifically request confirmation from the first party member that the services were delivered. The Platform may also or alternatively use location tracking, e.g., geo-tracking on the first party member's phone, showing that the first party member was at the second party's location for a certain period of time.

The Platform may then release funds from the account, e.g., escrow account and transfer them into the second party's account, e.g., digital wallet. The Platform may then send payment notification to the first party and the second party as well as the first party member.

As described in the previous embodiment, determining an adequate amount of funds to be kept on hand to ensure that the process isn't hindered, may be a difficult task. The first party wouldn't want to place more funds in an account then need be, but if the funds are insufficient, then the ability for their members to receive care could be stifled. In addition to the ongoing manual monitoring and management, there is included in another embodiment, a more automated and intelligent approach, e.g., a rules-based engine that can forecast future care based on historical claim details.

In another embodiment of the present invention there is a claims analytics engine. The embodiment uses a distributed computing network, with a distributed operating system, running a distributed ledger application with smart contracting functionality, medical billing claims processing engine and storage functionality, with business logic and rules engine (collectively, “Platform”).

As claims are submitted, processed, and paid through the Platform between two parties, the data related to those claims, e.g., parties involved, services delivered, amount billed, amount paid, and frequency of service, are stored on the Platform. Using analytical analysis, artificial intelligence, and machine learning, the amounts of future claims can be deduced. This information can be used to determine the amount required under a smart contract for a first party to maintain in their account, e.g., digital wallet, to support the escrow requirements that were previously defined.

In yet another embodiment of the present invention there is Master Patient Index with an API library. The embodiment includes a distributed computing network, with a distributed operating system, running a distributed ledger application with smart contracting functionality, medical billing claims processing engine and storage functionality, with business logic and rules engine, and a master index and national identifier that indexes patient record identifiers and API associated with each location where a patient's record exists (collectively, “Platform”).

A Master Patient Index with an associated API library serves as the translation and standardization hub for all the disparate instances of a patient's record that exist across all care locations. A master patient index and API library serves to index all instances of a patients record across the care continuum, enabling ubiquitous on demand access to the patient's entire medical record. This can support the dynamic creation of the longitudinal record that was previously discussed. The embodiment may enable the storage and indexing of all patient record identifiers that exist across every location where a patient's record exists. Each provider location typically utilizes a unique medical record number. The embodiment consumes these numbers and indexes them to the patient's national identifier that is also stored on the Platform. In conjunction with the identifiers from each location where the patient's record exists, is a link to all the APIs for the remote provider systems where the patient's records reside. When the patient grants access on the Platform to their medical record information, via a native or third-party platform application, the Platform is able to utilize this master patient index with its associated API library to query each and retrieve the patient's medical information to use it as the patient had approved.

In another embodiment of the present invention there is a “many to many” contracting method. The embodiment includes a distributed computing network, with a distributed operating system, running a distributed ledger application with smart contracting functionality, medical billing claims processing engine and storage functionality, with business logic and rules engine, and a master index that indexes patient record identifiers and API associated with each location where a patient's record exists, and functionality to support many-to-many relationships through a single or limited number of actual contracts with dynamic pricing and provider access management (collectively, “Platform”).

The embodiment enables payers to create an account on the platform, define one or more rates that they are willing to pay for healthcare services, and then engage under a contract on the Platform that applies the one or more rates they have defined for the payment of services that they received on the Platform. The embodiment further enables providers to create an account on the platform, define the one or more rates that they are willing to accept for healthcare services, and then engage under a contract on the Platform that applies the one or more rates they have defined for services that were delivered on the Platform. The one or more rates that were entered by each party, and thus the price matching algorithm that determines whether or not the parties are “in network” or “out of network” to each other.

For example, if a provider inputs that they won't accept less than 150% of Medicare rates and a payer inputs that they won't pay more than 130% of Medicare rates, then that provider would not be in-network for that payer's members.

A provider only needs to create one account, define their one or more rates, and participate under ONE contract, but that effectuates relationships between MANY payers under that one contract, with the in-network determinant being the one or more rates that were entered by the payer and the provider. This also applies for the payer only needing to create one account, defining their one or more rates, and participating under ONE contract as well.

In another embodiment of the present invention, there is provided a Dynamic Provider Directory. The embodiment includes a distributed computing network, with a distributed operating system, running a distributed ledger application with smart contracting functionality, medical billing claims processing engine and storage functionality, with business logic and rules engine, and a master index that indexes patient record identifiers and API associated with each location where a patient's record exists, with functionality to support many-to-many relationships through a single or limited number of actual contracts with dynamic pricing and provider access management, and dynamic price matching determinants to trigger in-network or out-of-network status displays for all participating providers and payers on the platform (collectively, “Platform”).

The embodiment may incorporate a web or app-based user interface that enables the user to view, sort, filter, search for providers who are participating under a contract on the platform and are in-network or out-of-network to their employer's health plan. The platform may use the rates that were entered by the employer and the provider, to determine if there is a match. If a match is determined, the provider would then be displayed as being in-network. If the provider is out-of-network based on these rates, and the employee and/or plan member wishes for the provider to become in-network, the employee can make the request via the provider directory user interface. This request would send a notification to the employer and the provider requesting that they each consider reassessing their rates to facilitate the in-network status.

In another embodiment of the present invention there is provided a process for network building. The embodiment includes a distributed computing network, with a distributed operating system, running a distributed ledger application with smart contracting functionality, medical billing claims processing engine and storage functionality, with business logic and rules engine, and a master index that indexes patient record identifiers and API associated with each location where a patient's record exists, with functionality to support many-to-many relationships through a single or limited number of actual contracts with dynamic pricing and provider access management, dynamic price matching determinants to trigger in-network or out-of-network status displays for all participating providers and payers on the platform, with the ability for multiple participants to define and create their own “sub-network”, e.g., filtered list of providers, and a streamlined process to perform provider outreach and ultimately usher them through the process of contracting on the platform (collectively, “Platform”).

The embodiment includes a Platform that is designed to create a network of contracted providers through a crowdsourced incentivization approach, that can be utilized by multiple payers simultaneously while affording them with the ability to white label their own network on the platform.

The process can be broken down into two phases: provider not yet registered and contracted on the platform, or provider is registered and contracted on the platform.

Where a provider is not yet registered and contracted on the Platform, the embodiment allows for provider outreach and onboarding. Provider outreach can be targeted to a specific user, e.g., an employer and/or their benefits consultant, that uploads a list of provider identifiers which their members have visited over some prior period of time, or specific providers that are identified for some other reason such as geographic location or specialty, or general, e.g., general marketing and advertising.

A provider may be uploaded into the platform using several provider information databases. The Platform may then fill in the provider's profile on the platform, establish the provider as a “Lead” to be targeted, determine the type of provider, e.g., hospital, health system, and medical group, and assign a “ranking” to the provider based on several factors, e.g., type of provider, number of times the provider has been uploaded, and geographic targeted areas. The Platform may then contact the provider, e.g., email the provider inviting them to join the platform. Based on their ranking, the Platform may then assign the provider to a specific Lead que in the onboarding process, e.g., direct outreach, fax outreach, email, and mail. The Platform can then track the provider Lead during the outreach process.

One method of outreach is through crowdsourcing or incentivization. The Platform supports the ability for channel partners to participate on the Platform to invite and usher providers through contracting, and to be incentivized to do so. A typical channel partner would be a healthcare sales professional who is currently selling to healthcare providers. They can leverage their existing relationships to earn incentives for inviting them, and subsequently contracting on the Platform. Channel partners can also invite other sales professionals to participate, effectively establishing a network of “sub partners” underneath them. A channel partner is also incentivized, albeit at a lower level, when a provider contracts after being invited by one of their sub partners.

During the process of registering, inputting one or more rates, and contracting, the platform may incorporate several functions that are designed to be informative to the providers in helping them recognize the impact to their business related to contracting on the Platform.

Based on the providers demographics, number of providers, and historical revenue data, the Platform may display a “contract impact analysis,” which demonstrates the estimated portion of their patient encounters and revenue that is attributed to self-funded employer health plans. This data is designed to help them assess the impact that contracting or not contracting on the platform will have on their business.

When the provider reaches the contracting stage of registration, as they enter their desired one or more rates for services, the Platform demonstrates the estimated percentage of employers that would deem them as in-network or out-of-network, based on the one or more rates they are about to commit to. This functionality enables the provider to name their desired one or more rates and helps to appreciate their market capture based on the one or more rates they have entered. The employers experience the same process, but from the opposite side of the Platform.

If the provider is registered and contracted on the Platform the network participation on the Platform is a transparent concept from the provider's perspective. Unless the provider is creating their own white labeled network, e.g., sub-network, the provider's involvement in a network or lack thereof, is not a factor to them. Once they are registered, have defined their one or more rates, and contracted, they are available and accessible to any payer on the platform who is willing to pay what the provider has requested.

Employers, benefits consultants, and even providers can create their own White Labeled Networks (“WLN”) on the Platform. A WLN is effectively a sub network on the Platform, operated with a different unique name, and possibly monetized through marking up the cost to the employer when they decide to use a WLN that is monetized by its creator. A WLN can include all providers on the platform, or the creator of the WLN can “filter” the providers based on many factors, e.g., quality, cost, utilization, geographic location, and specialty.

In yet another embodiment of the present invention there is provided a process for Provider Credentialing. The embodiment includes a distributed computing network, with a distributed operating system, running a distributed ledger application with smart contracting functionality, with business logic and rules engine, and a master index that index of provider information (collectively, “Platform”).

The embodiment enables providers to create an account on the platform and directly input, enter, upload, and store their sensitive credentialing information. In the process of entering this information, if the category of item typically requires “direct source verification”, e.g., information must be verified directly with the state or their malpractice carrier, then they can enter the information on the Platform to obtain this direct source verification, e.g., grant access through an API lookup. A smart contract on the Platform or multiple smart contracts with different rules can determine the credentialing requirements to grant the provider an “approval.” For example, a hospital may have some credentialing requirements, but a payer may other requirements. Both the provider and the other party agree to participate in one or more smart contracts. With the click of a button, the provider sees their approval or denial. If a denial, where the deficiency lies is also shown. The other party also sees the providers approval or denial and if a denial, where the deficiency lies. No provider information was conveyed to the other party. The provider remains in control of their information securely on the platform. The one or more smart contracts that were agreed upon by both parties determines the provider's credentialing status. In addition, this approach not only automates this process and affords the benefits listed above, but also centralizes it to make the process simpler for all parties. The provider doesn't have to repeat the credentialing process for every hospital or payer with which they are affiliated with and the payer doesn't have to repeat this process for every provider. When this process is integrated with many of the functions previously defined in the Platform, this automated credentialing status can also be used to determine provider in and/or out of network status as well.

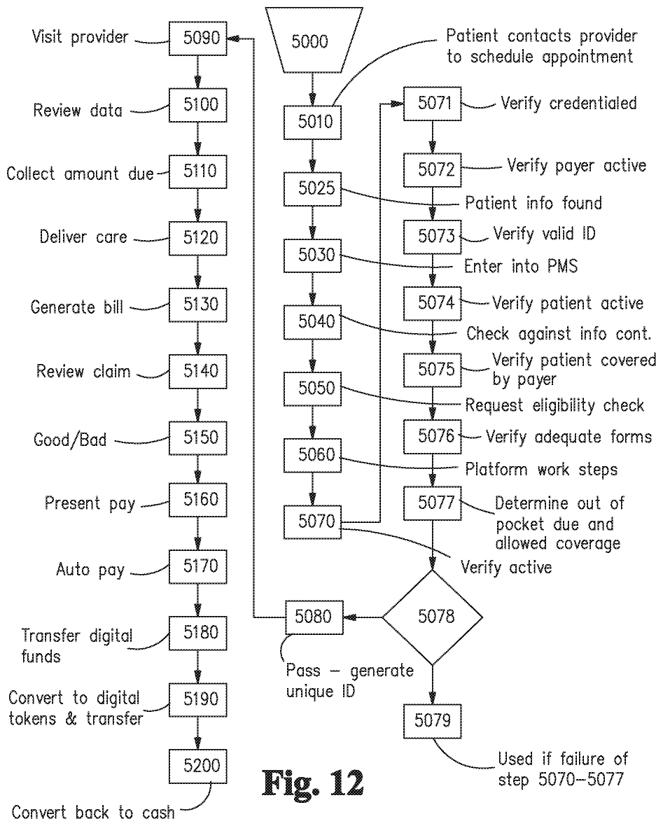

Referring now to FIG. 12, there is shown an embodiment of a Method and Process to Facilitate Automated Electronic Claims Processing and Payment. The invention employs a novel method and technique for conducting business that delivers tangible value in the form of cost and time savings to payors and increased revenue and timeliness of payment to providers, by facilitating an automated electronic medical billing claims processing and digital payment through the Platform.

Step 5000, an individual (“Patient”) who is covered under a contracted relationship between a payor and a provider on the Platform, requires services from the provider. Step 5010, the Patient contacts the provider to schedule an appointment. Step 5020, the provider receives the Patients information in preparation for the visit. Step 5025, the Patient may provide their full name, address, date of birth, social security number, and insurance information. Step 5030, the provider may input this information into their practice management system (“PMS”). Step 5040, the PMS may already contain a profile related to this specific payor, in the same fashion as it would contain insurance company profiles as payors. This is because the provider and this patient's payor had already established a network relationship and entered into a contractual relationship via the Platform. Step 5050, the provider may request an eligibility check through their PMS, on the Platform, or directly through the Platform.

Step 5060, the Platform may undertake a series of logical steps that includes, but is not limited to, the following and may require each step to be validated to continue to the next. Step 5070, verification that provider group account is active. Step 5071, verification that individual provider is credentialed. Step 5072, verification that the payor account is active. Step 5073, verification that the provider group and the payor have an active network relationship and valid contractual agreement. Step 5074, verification that the Patient account is active. Step 5075, verification that the Patient is actively covered under the payors account. Step 5076, verification of adequate funds in the payor's account. Step 5077, determination of the Patient's coverage amounts and out of pocket due for the visit. Step 5078, if any of the required Steps 5070-5077 fails, then go to Step 5079, denial of approval, else go to Step 5080.

Step 5080, if all the above required verifications pass, then the Platform may generate a unique identifier related to this event and stores it on the Platform. The Platform may then transmit one or more communications, including a standard EDI file to the providers PMS confirming this verification. Step 5090, the patient then visits the provider to receive care. Step 5100, at check-in, the provider's staff may view the entries that were electronically posted to the provider's PMS through the EDI file from the Platform. Step 5110, the information may contain the amount that is due by the Patient in relation to this visit, and the provider may collect these funds.

Step 5120, if the provider delivers care to the patient. Step 5130, the provider may then generate an invoice, e.g., an electronic billing claim on the PMS and submit it through an interface to the Platform. Step 5140, the Platform receives the claim, verifies the validity of the information using data such as the prior eligibility check. Step 5150, the Platform may then perform a series of validity checks using the rules engine. Step 5160, if the claim is determined to be acceptable, then the claim may be presented to the payor for review and payment. Step 5170, the payor can opt for auto payment of claims vs. manual review. Step 5180, based on the amount of funds required to pay the claim, the payor may transfer funds to an account, e.g., their digital wallet, on the Platform. Step 5190, the funds may be seamlessly and automatically converted to digital tokens, and the tokens may be immediately transferred to a provider account, e.g., digital wallet of the provider. Step 5200, the digital tokens may be automatically converted to back to currency, e.g., commodity or fiat, and deposited into the provider's account.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of building a network of users, the method comprising:
providing a platform, the platform having a distributed ledger, a web-based portal being configured for at least one connection, a first computer system in connection with the web-based portal, a second computer system having an application programming interface, the second computer in connection with the web-based portal, a session controller in connection with the web-based portal, at least one web-based engine in connection with the web-based portal, and at least one web-based database in connection with the web-based portal;
inputting at least one first user having at least one first user information on the platform;
saving the at least one first user information on the platform;
querying the platform for at least one second user;
inviting the at least one second user to join the network of users;
generating at least one first communication on the platform to facilitate the invitation;
requesting an acceptance of the invitation from the at least one second user; and
sending at least one notification to the at least one first user.

2. The method of claim 1, wherein the first user is a payor or a provider.

3. The method of claim 1, wherein the method further includes:
determining if the at least one second user has an account on the platform;
requesting the at least one second to create an account on the platform;
resending the request to the at least one second user to create an account on the platform after a predetermined time; and
notifying the at least one first user of a status of the at least one second user.

4. The method of claim 3, wherein the status of the at least one second user is an accept or a reject.

5. The method of claim 3, wherein the predetermined time is seventy-two hours.

6. The method of claim 1, wherein the at least one first communication is sent to at least one of the at least one first user, the at least one second user, and an administrator of the platform.

7. The method of claim 1, wherein the method further includes:
generating at least one second communication; and
adding the at least one second user to a first user account.

8. The method of claim 5, wherein the at least one second communication is an invitation to the at least one second user to create a provider account on the platform.

9. A method of facilitating direct contracting, the method comprising:
providing a platform having a distributed ledger, a web-based portal being configured for at least one connection, a first computer system in connection with the web-based portal, a second computer system having an application programming interface, the second computer in connection with the web-based portal, a session controller in connection with the web-based portal, at least one web-based engine in connection with the web-based portal and at least one web-based database in connection with the web-based portal;
establishing a relationship between a first user and a second user, the first user and the second user on the platform;
selecting a first user profile or a second user profile from a list on the platform;
reviewing at least one contract associated with the first user profile or the second user profile; and
selecting at least one of the at least one contract.

10. The method of claim 9, wherein the method further includes:
requesting at least one new contract between the first user and the second user;
accepting the at least one new contract; and
notifying at least one of the first user and the second user.

11. The method of claim 9, wherein the method further includes requesting the platform to create a project between the first user and the second user.

12. The method of claim 9, where the first user is a payor or a provider.

13. The method of claim 11, wherein the platform further includes an administrator to facilitate the creation of the project.

14. A system for managing information, the system comprising:
a distributed ledger;
a web-based portal being configured for at least one connection;
a first computer system in connection with the web-based portal;
a second computer system having an application programming interface, the second computer in connection with the web-based portal;
a session controller in connection with the web-based portal;
at least one web-based engine in connection with the web-based portal; and
at least one web-based database in connection with the web-based portal.

15. The system of claim 1, wherein the first computer system includes a global network of interconnected computers and the second computer system includes a National Provider Identification system.

16. The system of claim 1, wherein the at least one web-based engine includes at least one of a business process rules engine and a claims processing engine.

17. The system of claim 1, wherein the at least one web-based database includes at least one of a clinical data repository and a healthcare services pricing database.
